(19) United States
(12) Plant Patent Application Publication
(10) Pub. No.: US 2010/0251448 P1
(43) Pub. Date: Sep. 30, 2010

(54) VARIETY OF CALIBRACHOA PLANT NAMED 'WESCADOPI'

(50) ;

(76) Inventor: Heinrich Westhoff, Sudlohn (DE)

Correspondence Address: THE WEBB LAW FIRM, P.C., 700 KOPPERS BUILDING, 436 SEVENTH AVENUE, PITTSBURGH, PA, 15219, US

(21) Appl. No.: 12/383,757

(22) Filed: Mar. 26, 2009

(51) Int. Cl. A01H 5/00

(52) U.S. Cl. PLT/413

(57) ABSTRACT

A new and distinct Calibrachoa plant named ‘Wescadopi’ that exhibits double-flowers and a compact growth habit.

### BOTANICAL CLASSIFICATION

Calibrachoa

### VARIETAL DENOMINATION

‘Wescadopi’

### BACKGROUND OF THE INVENTION

The present invention comprises a new and distinct cultivar of Calibrachoa known by the varietal name ‘Wescadopi’. The new variety was discovered in Südlohn, Germany in August of 2007. The new variety has been vegetatively asexually reproduced via cuttings, and the first date of asexual reproduction was in 2007/2008 in Südlohn, Germany. The new variety is the result of a planned breeding program cross between unpatented Calibrachoa plant MiniFamous Double Pink (female parent) and unpatented Calibrachoa plant Seedling 06P942 (male parent). The purpose of the breeding program was to develop a double-flowering Calibrachoa variety. The new variety exhibits similar flower color and shape with its female parent, but exhibits smaller flowers and more compact plants than both parents. ‘Wescadopi’ exhibits a similar foliage color, flower bud size, and its double-flowering characteristic with Calibrachoa plant ‘MiniFamous Double Dark Pink’, (unpatented), but the new variety has smaller flowers that are lighter pink in color, less filled and unveined flowers, and smaller leaves (3.2×0.7 cm vs. 4.0×1.1 cm) when compared to ‘MiniFamous Double Dark Pink’. Further, the new variety differs from ‘MiniFamous Double Dark Pink’ because it has 5 petals inside the calyx, while ‘MiniFamous Double Dark Pink’ has 10-14 petals inside the calyx. The new variety has been trial and field tested and has been found to retain its distinctive characteristics and remain true to type through successive propagations. The following characteristics also distinguish ‘Wescadopi’ from other Calibrachoa varieties known to the breeder:
1. Compact growth habit;
2. Double-flowering pink flowers;
3. Elliptic-shaped and small-sized leaves;
4. Small-sized flowers without veins; and
5. Contrasting foliage and flower colors.

### DESCRIPTION OF THE DRAWINGS

The accompanying photographic drawings illustrate the new variety, with the color being as nearly true as is possible with color illustrations of this type:

FIG. 1 is a view of an entire plant of the new variety; and

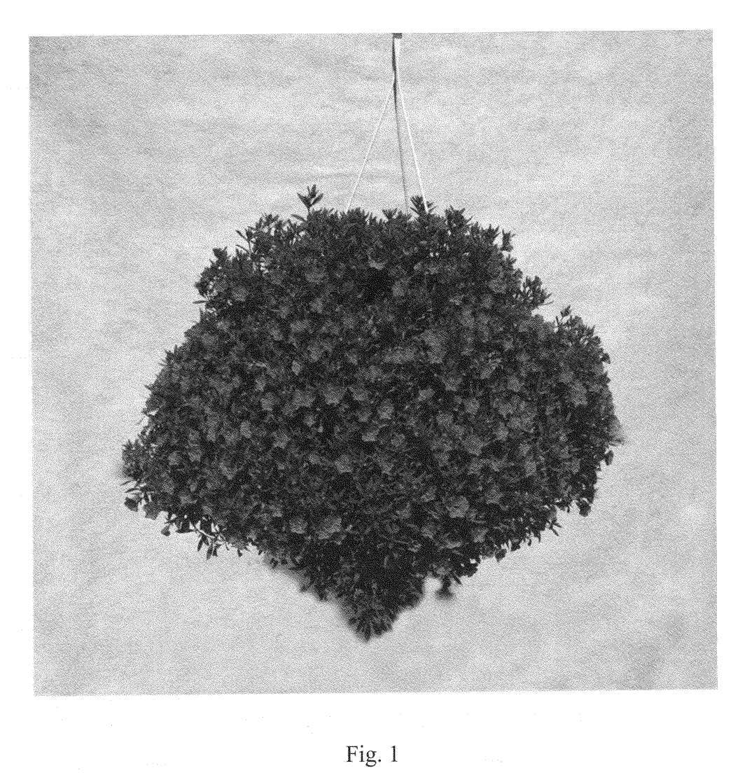

FIG. 2 is close-up photograph of a flower of the new variety.

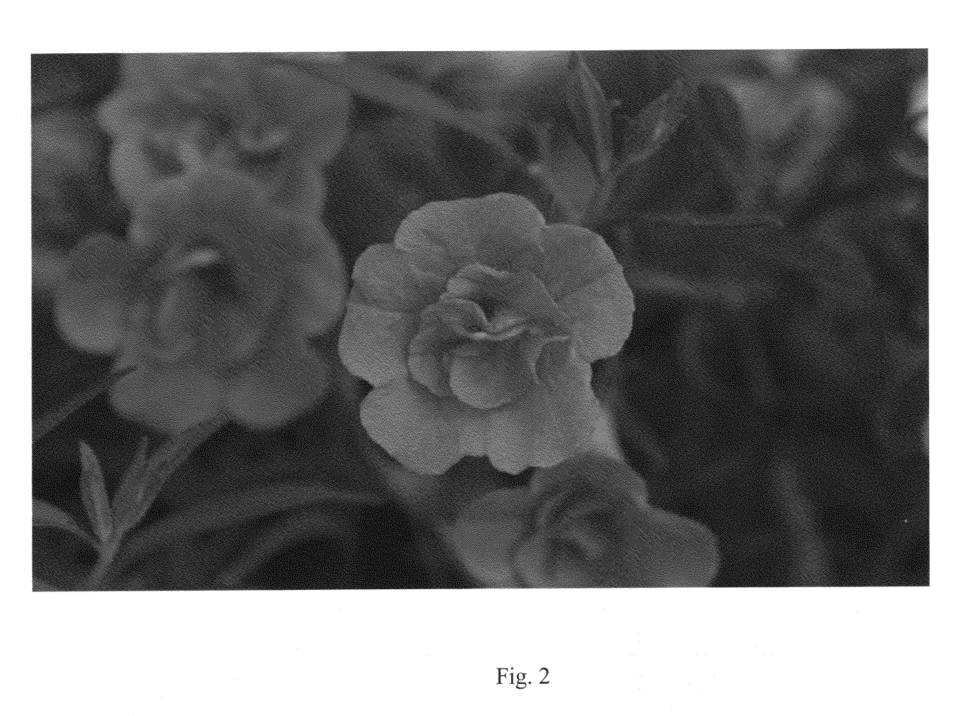

### DESCRIPTION OF THE PLANT

The following detailed description sets forth the characteristics of 25 week old ‘Wescadopi’ plants grown inside a greenhouse in 12 cm pots and baskets in Südlohn, Germany. The average day temperature was 20-25° C., and the average night temperature was 16-18° C. The color readings were taken indoors under natural light. Color references are primarily to the 2001 R.H.S. Colour Chart of The Royal Horticultural Society of London.
Plant:
   Time to initiate roots.—10-14 days at about 20° C.
   Time to develop roots.—21 days at about 20° C.
   Rooting habit.—Fine, fibrous, and well-branched.
   Root color.—158A.
   Form.—Compact and spherical.
   Branching habit.—Very good; at the base and every node.
   Height (from soil to top of flowers).—Pot grown plants: 12-15 cm. Basket grown plants: 24 cm.
   Plant diameter.—45-52 cm.
   Vigor.—Very strong.
   Lateral branches.—Form: Compact and spherical. Color: 144A. Texture: Dull, short pubescence. Diameter: About 1.0 mm. Internode length: About 0.6-1.5 cm. Quantity: At every node.
   Foliage.—Arrangement: Alternate and single. Number of leaves per branch: Plentiful; approximately 70-90 leaves per branch. Size of leaf: Length: About 3.2 cm. Width: About 0.7 cm. Shape of leaf: Narrow and elliptic. Shape of apex: Acute. Shape of base: Attenuate. Texture (upper and lower leaf surfaces): Dull. Aspect: Flat. Margin description: Entire. Color: Young leaves: Upper surface: 146A. Lower surface: 146B. Mature leaves: Upper surface: 147A. Lower surface: 147B. Petiole: Not applicable, leaves taper to the petiole. Veins: Color: Upper surface: 147B. Lower surface: 147C.
Flower:
   Natural flowering season.—From April to the end of autumn in Germany.
   Flower type and habit.—Single flowers near the top of branches.
   Number of flowers per plant.—Numerous; approximately 30-50 flowers per plant and 3-4 opened flowers per branch when grown in a 12 cm pot.
   Flower description.—Five petals are fused and form a usual Calibrachoa corolla. In the center of the corolla (the throat), the stamens are transformed into petals. The new variety has 5 small petals in the center that form a filled double flower.
   Corolla.—Diameter: About 2.3 cm. Flower tube length: About 1.5 cm. Flower tube diameter at distal end: About 0.63 cm. Flower tube diameter at proximal end: About 2.5 mm. Petal length from throat: Corolla petals: About 0.9 cm. Inner petals: About 0.38 cm. Petal width: Corolla petals: About 1.2 cm. Inner petals: About 1.0 cm. Petal quantity: Corolla petals: Five petals fused. Inner petals: Filled with 5 small petals. Petal apex shape: Corolla petals: Obtuse. Inner petals: Emarginate. Texture (upper and lower leaf surfaces): Smooth. Color: When opening: Upper surface: N74A. Lower surface: N74C. Fully opened: Upper surface: N74B. Lower surface: 75A to 75B. Flower throat (inside): 8A to 8D. Flower throat, vein: 166A. Flower tube (outside): 160C. Flower tube, vein: 166A. Petal color, fading to: N74B.
   Peduncle.—Length: About 1.0 cm. Diameter: About 0.9 mm. Color: 144A. Orientation: Acutely angled, holding all flowers outwards in optimum positions. Strength: Flexible.
   Bud.—Shape: Oblanceolate. Length: About 1.5 cm. Diameter: About 0.4 cm. Color: Base: 145C. Middle: 145C. Tip: 77C to 77D.
   Calyx.—Description: Salverform, filled calyx with double-flowering. Length: About 2.3 cm. Diameter: About 2.3 cm. Sepal shape: Five lanceolate-shaped (3 long, 2 short) sepals fused at the base. Sepal margin: Entire. Sepal texture (upper and lower leaf surfaces): Dull, pubescence present. Sepal color: Upper surface: 147B. Lower surface: 147B.
   Disease resistance.—None observed to date.
   Fragrance.—None.
   Weather tolerance.—Good.
   Lasting quality.—Long lasting; 8-14 days, depending on environmental conditions.
   Seed production.—None observed to date.
Reproductive organs:
   Stamens.—Absent because stamens transformed to petals.
   Pistils.—Number: One to three. Length: About 7.0 mm. Styles: Length: About 4.5 to 5.0 mm. Color: 144D. Stigma: Shape: Ovate. Color: 144A. Ovary color: 144C.

I claim:

1. A new and distinct variety of Calibrachoa plant named ‘Wescadopi’ substantially as shown and described herein.
